(19) United States
(12) Patent Application Publication
(10) Pub. No.: US 2014/0067085 A1
(43) Pub. Date: Mar. 6, 2014

(54) PROSTHETIC SLEEVE AND CUFF ASSEMBLIES

(71) Applicants: Jeffrey Lee Doddroe, Washington Court House, OH (US); Lonnie Lavern Nolt, Washington Court House, OH (US); Larry Gail Rowe, JR., Orient, OH (US); James M. Colvin, Hilliard, OH (US)

(72) Inventors: Jeffrey Lee Doddroe, Washington Court House, OH (US); Lonnie Lavern Nolt, Washington Court House, OH (US); Larry Gail Rowe, JR., Orient, OH (US); James M. Colvin, Hilliard, OH (US)

(21) Appl. No.: 14/019,125

(22) Filed: Sep. 5, 2013

Related U.S. Application Data
(60) Provisional application No. 61/696,929, filed on Sep. 5, 2012.

(51) Int. Cl. A61F 2/80

(52) U.S. Cl. CPC A61F 2/80
USPC 623/36

(57) ABSTRACT

Prosthetic cuffs and prosthetic sleeve and cuff assemblies are provided.

### CROSS-REFERENCE TO RELATED APPLICATIONS

This application claims priority from U.S. Provisional Patent Application No. 61/696,929, filed on Sep. 5, 2012, which is incorporated by reference herein in its entirety.

### BACKGROUND

Amputees retaining a residual limb often utilize prosthetic limbs attached to the residual limb via a prosthetic socket. Prosthetic sockets are often attached to the residual limb through a differential pressure, wherein the interior of the prosthetic socket surrounding the residual limb is maintained at a sub-atmospheric pressure.

Some prosthetic sockets maintain a sub-atmospheric pressure within a prosthetic socket via a sleeve device that is substantially impermeable to air. However, such sleeves are prone to perforation when impacted against hard objects, which results in loss of the sub-atmospheric pressure within the prosthetic socket. Damage to the sleeve device often requires costly replacement of the sleeve. Additionally, unless an amputee happens to have a replacement sleeve on hand, proper retention of the prosthetic socket and thus prosthetic limb is not likely.

What is needed is a prosthetic sleeve and cuff assembly that permits an amputee to quickly remedy a perforation and loss of sub-atmospheric pressure, and which also provides additional protection and cushioning against impact forces that may cause perforations.

### SUMMARY

In one embodiment, a prosthetic cuff for use with a prosthetic socket is provided, the prosthetic cuff comprising: a tubular body portion comprising an inner surface and an outer surface; wherein at least a portion of the inner surface includes a tacky elastomer; wherein at least a portion of the inner surface is configured to contact a prosthetic socket; and wherein at least a portion of the outer surface includes a tacky elastomer.

In another embodiment, a prosthetic cuff for use with a prosthetic socket is provided, the prosthetic cuff comprising: a tubular body portion comprising an inner surface, an outer surface, and an interior section between the inner surface and the outer surface; wherein at least a portion of at least one of the inner surface and the interior section comprises a fabric reinforcement layer; wherein at least a portion of the inner surface is configured to contact a prosthetic socket; and wherein at least a portion of the outer surface includes a tacky elastomer.

In another embodiment, a prosthetic cuff and sleeve assembly for use with a prosthetic socket is provided, the prosthetic cuff and sleeve assembly comprising: a cuff comprising a cuff tubular body portion, wherein the cuff tubular body portion comprises a cuff inner surface and a cuff outer surface; wherein at least a portion of the cuff inner surface is configured to contact a prosthetic socket; and wherein at least a portion of the cuff outer surface includes a tacky elastomer; and a sleeve comprising a sleeve tubular body portion, wherein the sleeve tubular body portion comprises a sleeve inner surface and a sleeve outer surface; wherein at least a portion of the sleeve inner surface includes a tacky elastomer.

### BRIEF DESCRIPTION OF THE DRAWINGS

The accompanying figures, which are incorporated in and constitute a part of the specification, illustrate various example apparatuses and systems, and are used merely to illustrate various example embodiments.

FIG. 1 illustrates an example arrangement of a prosthetic sleeve and cuff assembly.

FIG. 2 illustrates a partial view of an example arrangement of a prosthetic sleeve and cuff assembly.

FIG. 3 illustrates a partial view of an example arrangement of a prosthetic sleeve and cuff assembly.

FIG. 4 illustrates a partial view of an example arrangement of a prosthetic sleeve and cuff assembly.

FIG. 5 illustrates a partial view of an example arrangement of a prosthetic sleeve and cuff assembly.

### DETAILED DESCRIPTION

The present embodiments describe prosthetic cuffs and prosthetic sleeve and cuff assemblies. For ease of understanding and to provide context, selected aspects of example prosthetic cuffs and prosthetic sleeve and cuff assemblies, as well as prosthetic sockets with which such assemblies may interact, are generally set forth below.

### Prosthetic Sockets

Many amputees retain at least a portion of the amputated limb. For example, an amputee may have a leg amputation just above the knee, leaving much of the femur and thigh intact. This intact portion is generally referred to as the residual limb.

Prosthetic limbs include a prosthetic socket. A prosthetic socket may be described as a hollowed structure configured to extend over the residual limb, with the interior hollowed cavity substantially mating with the exterior size and contour of the residual limb and any interface materials. The residual limb and any interface materials are inserted into the prosthetic socket, which in turn is attached to any of various prosthetic devices (e.g., a prosthetic foot). The prosthetic socket may be formed of any of a variety of materials, including polymer(s), metal(s), composite material(s), or a combination of materials. For example, the prosthetic socket may be a hard polymer material.

Maintaining a residual limb within a prosthetic socket can be achieved by creating a sub-atmospheric pressure within the prosthetic socket. That is, the air pressure within the prosthetic socket may be reduced to a level that is lower than the air pressure outside of the socket, thus creating a differential pressure. The greater air pressure outside of the prosthetic socket acts to force and maintain the prosthetic socket into place on the residual limb. However, to maintain a differential pressure, a substantially air impermeable seal must be made between the interior of the prosthetic socket and the exterior air.

### Prosthetic Sleeves

Prosthetic sleeves are substantially tubular devices configured to be placed over the junction of the brim of a prosthetic socket (the proximal-most portion of the prosthetic socket) of a prosthetic limb, and the residual limb itself. For example, the prosthetic sleeve may be configured to create a substantially air impermeable seal about the brim of the prosthetic socket, to assist in maintaining a sub-atmospheric air pressure within the prosthetic socket.

The prosthetic sleeve's tubular body portion includes a radially inner surface and a radially outer surface. Prosthetic sleeves may be comprised of any of a variety of materials, including a thermoplastic elastomer, urethane, silicone, thermoplastic urethane, and thermoplastic silicone. In one embodiment, the prosthetic sleeve is comprised of a combination of materials. In another embodiment, the prosthetic sleeve comprises a silicone. In one embodiment, the prosthetic sleeve comprises a silicone having a hardness between about Shore OO 10 and about Shore OO 60 (as measured according to ASTM D2240-05(2010) Standard Test Method for Rubber Property 8212—Durometer Hardness). In another embodiment, the prosthetic sleeve comprises a silicone having an elongation between about 700% and about 1400% (as measured according to ASTM D412-06ae2 Standard Test Methods for Vulcanized Rubber and Thermoplastic Elastomers—Tension). In one embodiment, the inner surface of the prosthetic sleeve comprises the same material that makes up the prosthetic sleeve. In another embodiment, at least a portion of the inner surface of the prosthetic sleeve comprises a tacky elastomer.

At least a portion of the outer surface of the prosthetic sleeve may comprise the same material that makes up the sleeve. In another embodiment, at least a portion of the outer surface of the prosthetic sleeve comprises a fabric layer. In one embodiment, the outer surface comprises a fabric layer to at least one of improve aesthetics, improve durability, and improve ease of use. In another embodiment, the outer surface of the prosthetic sleeve comprises a fabric layer to resist clinging between the prosthetic sleeve and items with which the prosthetic sleeve may come in contact (e.g., furniture or clothing). Fabrics used in the fabric layer may comprise a fabric with very high stretch and low modulus in both the circumferential and the length directions. The high stretch of the fabric may allow the prosthetic sleeve to fit a wide range of residual limb circumferences comfortably, and may also allow for a good range of motion at residual limb joints with little restriction or resistance. One example fabric may be a single jersey, plain knit construction seamlessly knit on a flat bed knitting machine composed of 93.4% microfiber nylon and 6.6% Spandex. Such fabric, when tested on a Zwick tensile test machine according to ASTM Standard D4964-96(2008)e2, Standard Test Method for Tension and Elongation of Elastic Fabrics (Constant Rate of Extension Type Tensile Testing Machine), using a 151b load cycled three times with modulus and stretch taken on the third outgoing cycle, may have a length stretch and 50% modulus in the range of about 140% to about 180% and about 0.40 lbf to about 0.80 lbf, respectively, and a circumferential stretch and 50% modulus in the range of about 170% to about 210% and about 0.18 lbf to about 0.30 lbf, respectively.

In one embodiment, the prosthetic sleeve comprises a substantially tubular shape with a tapered diameter. The prosthetic sleeve may comprise a tapered diameter to substantially match the contour of the prosthetic socket and residual limb over which the prosthetic sleeve extends. In one embodiment, the prosthetic sleeve is between about 8 in. and about 24 in. in length. In another embodiment, the prosthetic sleeve is between about 12 in. and about 20 in. in length. In another embodiment, the prosthetic sleeve is between about 14 in. and about 18 in. in length. Lengths shorter than 8 in. and longer than 24 in. are contemplated, depending on the application.

The prosthetic sleeve may be subject to extreme impact forces, sandwiched between the prosthetic socket and structures with which the prosthetic socket may come into contact, including door frames, furniture, tables, etc. Generally speaking, when a prosthetic socket contacts any of various hard items, the prosthetic sleeve risks becoming perforated. Such perforation of a prosthetic sleeve can cause loss of sub-atmospheric pressure within the interior of the prosthetic socket. Loss of sub-atmospheric pressure within the interior of a prosthetic socket can undesirably allow the prosthetic socket to move relative to the residual limb. In one embodiment of the present invention, prosthetic sleeves are configured to assist in the cushioning of contact between the prosthetic socket and any of various hard items as noted above.

### Prosthetic Cuffs

The prosthetic cuff is a substantially tubular device configured to be placed over the junction of the brim of a prosthetic socket (the proximal-most portion of the prosthetic socket) of a prosthetic limb, and the residual limb itself, radially inward of the prosthetic sleeve. In one embodiment of the present invention, the prosthetic cuff is directly contacted about the brim portion of a prosthetic socket, while the prosthetic sleeve is positioned radially outwardly of the prosthetic cuff. In one embodiment, the prosthetic cuff is configured to create a substantially air impermeable seal about the brim of the prosthetic socket, so as to assist in maintaining a sub-atmospheric air pressure within the prosthetic socket. In another embodiment, the prosthetic cuff is configured to interface with the prosthetic sleeve to create a substantially air impermeable seal between the prosthetic cuff and the prosthetic sleeve.

In one embodiment, the residual limb dimensions will dictate the exact placement of the prosthetic sleeve and prosthetic cuff, as well as the relative position of the prosthetic sleeve to the prosthetic cuff. The length of the residual limb as well as the diameter and taper of the residual limb can all affect the placement of the prosthetic sleeve relative to the prosthetic cuff. For example, if a residual limb is an amputee's thigh, then the brim portion of the prosthetic socket may end too closely to the amputee's groin to allow centering of the prosthetic cuff or prosthetic sleeve over the brim portion. In such a situation, more of the prosthetic cuff or prosthetic sleeve may extend below the brim portion than above the brim portion.

The prosthetic cuff's tubular body may include a radially inner surface and a radially outer surface. Prosthetic cuffs may comprise any of a variety of materials, including a thermoplastic elastomer, urethane, silicone, thermoplastic urethane, and thermoplastic silicone. In one embodiment, the prosthetic sleeve is comprised of a combination of materials. In another embodiment, the prosthetic cuff comprises a silicone. In one embodiment, the prosthetic cuff comprises a silicone having a hardness between about Shore A 2 and about Shore A 15 (as measured according to ASTM D2240-05(2010) Standard Test Method for Rubber Property 8212—Durometer Hardness). In another embodiment, the prosthetic cuff comprises a silicone having an elongation between about 500% and about 1100% (as measured according to ASTM D412-06ae2 Standard Test Methods for Vulcanized Rubber and Thermoplastic Elastomers—Tension).

In one embodiment, the inner surface of the prosthetic cuff comprises the same material that makes up the prosthetic cuff. In another embodiment, at least a portion of the inner surface of the prosthetic cuff comprises a tacky elastomer. The tacky elastomer inner surface of the prosthetic cuff may assist in creating a substantially air impermeable seal about the brim portion of the prosthetic socket so as to maintain a substantially sub-atmospheric pressure within the prosthetic socket when installed on the residual limb.

In one embodiment, at least a portion of the inner surface of the prosthetic cuff comprises a fabric layer. In one embodiment, the inner surface fabric layer may assist in protecting the prosthetic cuff from abrasive forces caused by the socket brim. In another embodiment, the inner surface fabric layer may act as a fabric reinforcement layer. In one embodiment, only a portion of the inner surface of the prosthetic cuff comprises a fabric layer. For example, the upper portion of the inner surface of the prosthetic cuff may comprise a fabric layer. In this embodiment, the fabric layer may be configured to contact the brim portion of the prosthetic socket, while the remaining tacky elastomer on the lower portion of the inner surface of the prosthetic cuff is configured to contact a non-brim portion of the prosthetic socket. In this manner, the fabric layer portion may act to resist abrasive forces caused by the socket brim while the tacky elastomer portion may seal against the outer prosthetic socket wall.

The prosthetic cuff may also comprise an embedded fabric material contained within an interior section of the cuff between the inner surface and the outer surface. That is, the prosthetic cuff may comprise a fabric within the wall of the prosthetic cuff's tubular body. In one embodiment, the embedded material is a mesh fabric material. In another embodiment, the embedded mesh reinforces the prosthetic cuff. In another embodiment, the embedded mesh reduces or prevents tearing of the prosthetic cuff while allowing the prosthetic cuff to comprise a tacky elastomer on its inner and outer surfaces. In one embodiment, the fabric embedded in the interior section of the cuff comprises Featherstretch by Rx Textiles. In another embodiment, the fabric is a mesh nylon stockinette material with 475% stretch.

The outer surface of the prosthetic cuff may comprise the same material that makes up the prosthetic cuff. In one embodiment, at least a portion of the outer surface of the prosthetic cuff comprises a tacky elastomer configured to interface with the inner surface of the prosthetic sleeve. The sleeve-cuff interface may comprise a substantially air impermeable interface so that air cannot pass through or between the sleeve and cuff

In one embodiment, the prosthetic cuff comprises a substantially tubular shape with a tapered diameter. The prosthetic cuff may comprise a tapered diameter to substantially match the contour of the prosthetic socket and residual limb over which prosthetic cuff extends. In one embodiment, the prosthetic cuff is between about 2 in. and about 10 in. in length. In another embodiment, the prosthetic cuff is between about 4 in. and about 8 in. in length. In another embodiment, the prosthetic cuff is between about 5 in. and about 7 in. in length. It is contemplated that the prosthetic cuff may have a length of less than 2 in. and greater than 10 in.

The prosthetic cuff may be subject to extreme impact forces as it is sandwiched between the prosthetic socket and structures with which the prosthetic socket may come into contact, including door frames, furniture, tables, etc. Generally speaking, when a prosthetic socket contacts any of various hard items, the prosthetic cuff risks becoming perforated. Such perforation of a prosthetic cuff can cause loss of the sub-atmospheric pressure within the interior of the prosthetic socket. Loss of the sub-atmospheric pressure within the interior of a prosthetic socket can undesirably allow the prosthetic socket to move relative to the residual limb. In one embodiment of the present invention, the prosthetic cuff is configured to assist in the cushioning of contact between the prosthetic socket and any of various hard items as noted above. In another embodiment, the layering of a prosthetic sleeve over the prosthetic cuff provides additional cushioning between the prosthetic socket and any of the various hard items against which it may impact.

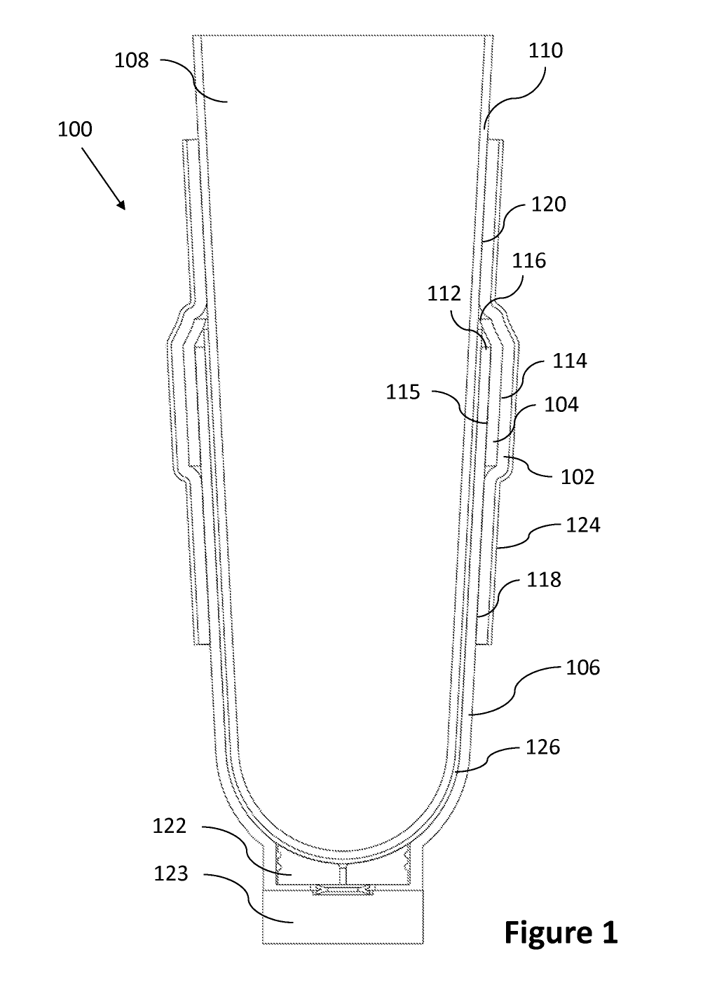

Having generally set forth selected aspects of prosthetic cuffs and prosthetic sleeve and cuff assemblies as contemplated herein, FIG. 1 illustrates a more particular example arrangement of a prosthetic sleeve and cuff assembly 100. Assembly 100 includes a sleeve 102 and a cuff 104. Assembly 100 additionally includes a prosthetic socket 106 into which an amputee's residual limb 108 is inserted. Assembly 100 may include a liner 110 placed over residual limb 108 for protecting residual limb 108 from prosthetic socket 106. Prosthetic socket 106 may comprise a brim portion 112, which is defined as the most proximal portion of prosthetic socket 106 and through which residual limb 108 is introduced into prosthetic socket 106.

Cuff 104 may comprise an outer surface comprising a tacky elastomer. Sleeve 102 may comprise an inner surface comprising a tacky elastomer. The cuff-sleeve interface 114 between cuff 104 and sleeve 102 may comprise an interface between two surfaces comprising a tacky elastomer. In one embodiment, cuff-sleeve interface 114 is substantially impermeable to air. In another embodiment, cuff-sleeve interface 114 is substantially impermeable to air having a differential pressure; that is, the air pressure on the outside of cuff-sleeve interface 114 is higher or lower than the air pressure on the inside of cuff-sleeve interface 114.

In one embodiment, cuff 104 is substantially tubular in shape. In one embodiment, cuff 104 extends about the periphery of brim portion 112. In one embodiment, cuff 104 contacts brim portion 112 creating a cuff-socket interface 115 that is substantially impermeable to air. In another embodiment, cuff-socket interface 115 is substantially impermeable to air having a differential pressure; that is, the air pressure on the outside of cuff-socket interface 115 is higher or lower than the air pressure on the inside of cuff-socket interface 115.

In one embodiment, cuff 104 contacts liner 110, creating a cuff-liner interface 116. At least a portion of liner 110 may comprise a tacky elastomer. In one embodiment, cuff-liner interface 116 is substantially impermeable to air. In another embodiment, cuff-liner interface 116 is substantially impermeable to air having a differential pressure; that is, the air pressure on the outside of cuff-liner interface 116 is higher or lower than the air pressure on the inside of cuff-liner interface 116.

In one embodiment, sleeve 102 is substantially tubular in shape. In one embodiment, sleeve 102 extends about the periphery of prosthetic socket 106. In one embodiment, sleeve 102 contacts prosthetic socket 106 creating a sleeve-socket interface 118 that is substantially impermeable to air. In another embodiment, sleeve-socket interface 118 is substantially impermeable to air having a differential pressure; that is, the air pressure on the outside of sleeve-socket interface 118 is higher or lower than the air pressure on the inside of sleeve-socket interface 118.

In one embodiment, sleeve 102 extends about the periphery of liner 110. At least a portion of liner 110 may comprise a tacky elastomer. In one embodiment, sleeve 102 contacts liner 110 creating a sleeve-liner interface 120 that is substantially impermeable to air. In another embodiment, sleeve-liner interface 120 is substantially impermeable to air having a differential pressure; that is, the air pressure on the outside of sleeve-liner interface 120 is higher or lower than the air pressure on the inside of sleeve-liner interface 120.

In one embodiment, assembly 100 comprises a prosthetic socket 106 configured to be held onto residual limb 108 via (at least in part) sleeve 102, such that sleeve-socket interface 118 and sleeve-liner interface 120 contribute to sleeve 102 holding prosthetic socket 106 into place relative to residual limb 108. In another embodiment, assembly 100 comprises a prosthetic socket 106 configured to be held onto residual limb 108 via (at least in part) cuff 104, such that cuff-sleeve interface 116 and cuff-socket interface 115 contribute to cuff 104 holding prosthetic socket 106 into place relative to residual limb 108. In another embodiment, both sleeve 102 and cuff 104 contribute to holding prosthetic socket 106 in place relative to residual limb 108.

In one embodiment, assembly 100 comprises a prosthetic socket 106 configured to be held onto residual limb 108 via (at least in part) a passive vacuum system comprising a valve 122. For example, valve 122 may be opened so as to allow air to pass from the interior of prosthetic socket 106 and through valve 122. Residual limb 108 may be inserted into prosthetic socket 106 thus causing at least a portion of the air within the interior of prosthetic socket 106 to evacuate, at which point valve 122 may be closed. As a result, the interior of prosthetic socket 106 may be at least substantially devoid of air after insertion of residual limb 108 and closing of valve 122, thereby creating a sub-atmospheric pressure within prosthetic socket 106. Sleeve 102 and cuff 104 may create a substantially air impermeable seal about brim portion 112 so as to maintain the sub-atmospheric pressure within prosthetic socket 106. Prosthetic socket 106 may be held in place relative to residual limb 108 as a result of the sub-atmospheric pressure within prosthetic socket 106 and atmospheric pressure outside prosthetic socket 106, which in turn creates a differential pressure.

In one embodiment, assembly 100 comprises a prosthetic socket 106 configured to be held onto residual limb 108 via (at least in part) an active vacuum system comprising valve 122. For example, valve 122 may be opened so as to allow air to pass from the interior of prosthetic socket 106 and through valve 122. Residual limb 108 may be inserted into prosthetic socket 106 thus causing at least a portion of the air within the interior of prosthetic socket 106 to evacuate, at which point a vacuum pump 123 operatively connected to valve 122 may be activated, further decreasing the air pressure within prosthetic socket 106, after which valve 122 may be closed. As a result, the interior of prosthetic socket 106 may be at least substantially devoid of air after insertion of residual limb 108, activation of vacuum pump 123 operatively connected to valve 122, and closing of valve 122, thereby creating a sub-atmospheric pressure within prosthetic socket 106. Sleeve 102 and cuff 104 may create a substantially air impermeable seal about brim portion 112 so as to maintain the sub-atmospheric pressure within prosthetic socket 106. Prosthetic socket 106 may be held in place relative to residual limb 108 as a result of the sub-atmospheric pressure within prosthetic socket 106 and atmospheric pressure outside prosthetic socket 106, which in turn creates a differential pressure.

In one embodiment, prosthetic cuff 104 is configured to extend about the periphery of brim portion 112, creating cuff-socket interface 115 and cuff-sleeve interface 116. Prosthetic cuff 104 creates a substantially air impermeable seal with the interior of prosthetic socket 106, such that the interior of prosthetic socket 106 is at a sub-atmospheric air pressure. In one embodiment, where prosthetic cuff 104 becomes perforated, at either cuff-socket interface 115 or cuff-sleeve interface 116, and where such perforation is not in communication with brim portion 112, the tacky elastomer inner surface of cuff 104 is configured to maintain a substantially air impermeable seal around the perforation in cuff-socket interface 115 and cuff-sleeve interface 116.

In one embodiment, sleeve 102 is oriented radially outward of prosthetic cuff 104 and is configured to extend about the periphery of brim portion 112 radially outward of cuff 104, creating cuff-sleeve interface 114, sleeve-socket interface 118, and sleeve-liner interface 120. Sleeve 102 creates a substantially air impermeable seal at each of cuff-sleeve interface 114, sleeve-socket interface 118, and sleeve-liner interface 120. In one embodiment, where sleeve 102 becomes perforated, the tacky elastomer inner surface of sleeve 102 is configured to maintain a substantially air impermeable seal around the perforation in each of cuff-sleeve interface 114, sleeve-socket interface 118, and sleeve-liner interface 120. In another embodiment, where both sleeve 102 and cuff 104 become perforated through a common perforation near brim portion 112 (e.g., a sharp object penetrates through both of sleeve 102 and cuff 104), air outside prosthetic socket 106 may enter the interior of prosthetic socket 106, thus undesirably destroying the sub-atmospheric pressure within prosthetic socket 106. In such a situation, one of sleeve 102 and cuff 104 may be rotated about an axis substantially aligned with residual limb 108, such that the perforation through sleeve 102 is no longer aligned with the perforation through cuff 104. Following the misalignment of the perforations through sleeve 102 and cuff 104, cuff-sleeve interface 114 may once again be substantially air impermeable and a sub-atmospheric pressure within prosthetic socket 106 may be restored. In another embodiment, sleeve 102 and cuff 104 may be moved relative to one another in any manner so as to misalign the perforation through each, including by rotating sleeve 102 relative to cuff 104, rotating cuff 104 relative to sleeve 102, translating (e.g., shifting upward or downward) sleeve 102 relative to cuff 104, or translating cuff 104 relative to sleeve 102.

In one embodiment, sleeve 102 may comprise a tubular body portion, comprising an inner surface and an outer surface. In one embodiment, at least a portion of sleeve 102′s inner surface includes a tacky elastomer. In one embodiment, at least a portion of sleeve 102′s outer surface comprises a fabric layer 124. Fabric layer 124 may be configured to provide at least one of an aesthetically pleasing exterior surface for sleeve 102, a further layer of protection against abrasion of sleeve 102, and a reduced friction interface between the exterior surface of sleeve 102 and other materials or clothing that an amputee may contact with sleeve 102 (e.g., clothing, furniture, etc.).

In one embodiment, liner 110 may comprise a substantially sock-like body portion, comprising an inner surface and an outer surface. In one embodiment, at least a portion of liner 110's inner surface comprises an elastomer. In one embodiment, at least a portion of liner 110's outer surface comprises a fabric layer 126. In one embodiment, fabric layer 126 extends along liner 110 where liner 110 contacts the interior of prosthetic socket 106. Liner 110's elastomer layer may extend proximally of prosthetic socket 106 and brim portion 112 along residual limb 108. Fabric layer 126 may be configured to provide at least one of a reduced friction interface between liner 110 and prosthetic socket 106 and a layer of protection against abrasion of liner 110. In practice, residual limb 108 may be covered with liner 110, after which residual limb 108 and liner 110 are inserted into prosthetic socket 106. In such an embodiment, the reduced friction interface between liner 110 and prosthetic socket 106 may assist in the insertion of residual limb 108 into prosthetic socket 106. The elastomer portion of liner 110 may extend upwardly along residual limb 108 and interface with sleeve 102 at sleeve-liner interface 120.

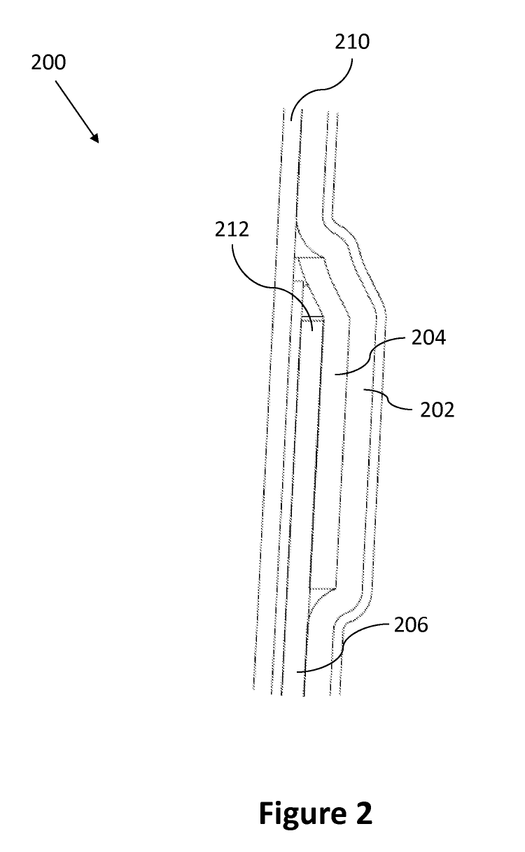

FIG. 2 illustrates a partial view of another example arrangement of a prosthetic sleeve and cuff assembly 200. Assembly 200 comprises a prosthetic sleeve 202, which is oriented radially outward of a prosthetic cuff 204, which is oriented radially outward of a prosthetic socket 206. Assembly 200 further includes a liner 210 oriented radially inward of prosthetic cuff 204 and prosthetic socket 206. Prosthetic socket 206 comprises a brim portion 212.

Prosthetic cuff 204 may comprise an elastomer material. Prosthetic cuff 204 comprises an inner surface and an outer surface, each of which may comprise a tacky elastomer.

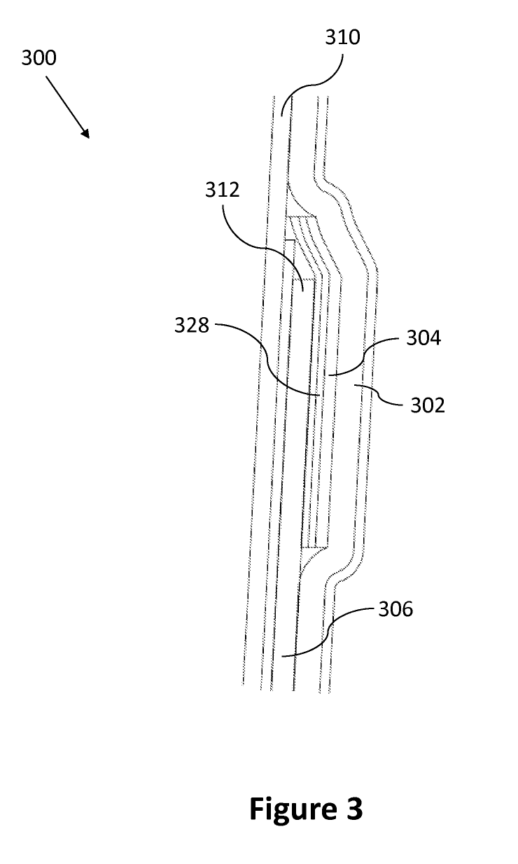

FIG. 3 illustrates a partial view of another example arrangement of a prosthetic sleeve and cuff assembly 300. Assembly 300 comprises a prosthetic sleeve 302, which is oriented radially outward of a prosthetic cuff 304, which is oriented radially outward of a prosthetic socket 306. Assembly 300 further includes a liner 310 oriented radially inward of prosthetic cuff 304 and prosthetic socket 306. Prosthetic socket 306 comprises a brim portion 312.

Prosthetic cuff 304 may comprise an elastomer material. Prosthetic cuff 304 comprises an inner surface and an outer surface, each of which may comprise a tacky elastomer. Prosthetic cuff 304 may comprise an interior section between the inner surface and the outer surface. In one embodiment, prosthetic cuff 304 comprises an embedded fabric reinforcement layer 328 oriented in the interior section. In one embodiment, fabric reinforcement layer 328 is configured to at least substantially prevent propagation of tears or perforations in prosthetic cuff 304.

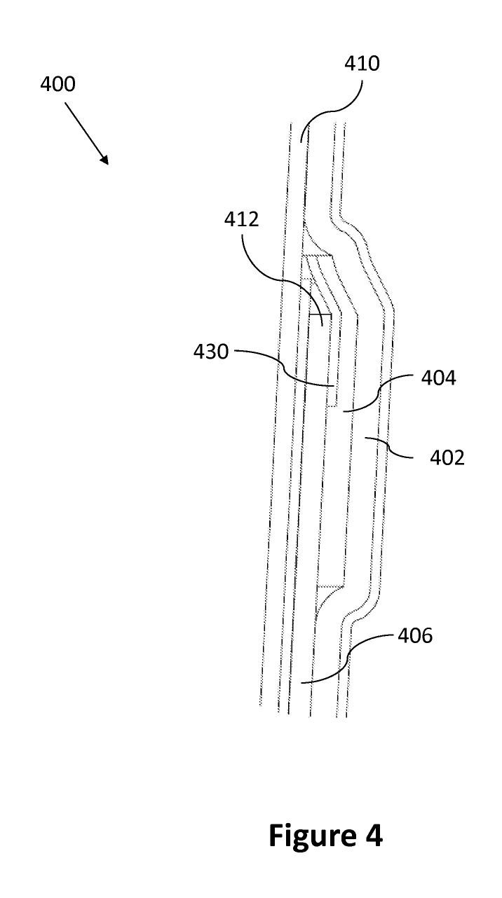

FIG. 4 illustrates a partial view of another example arrangement of a prosthetic sleeve and cuff assembly 400. Assembly 400 comprises a prosthetic sleeve 402, which is oriented radially outward of a prosthetic cuff 404, which is oriented radially outward of a prosthetic socket 406. Assembly 400 further includes a liner 410 oriented radially inward of prosthetic cuff 404 and prosthetic socket 406. Prosthetic socket 406 comprises a brim portion 412.

Prosthetic cuff 404 may comprise an elastomer material. Prosthetic cuff 404 comprises an inner surface and an outer surface, at least a portion of each of which may comprise a tacky elastomer. In one embodiment, prosthetic cuff 404 comprises a fabric reinforcement layer 430. In one embodiment, fabric reinforcement layer 430 is configured to contact prosthetic socket 406. In another embodiment, fabric reinforcement layer 430 is configured to contact brim portion 412. In another embodiment, fabric reinforcement layer 430 is configured to at least partially prevent or mitigate abrasion of prosthetic cuff 404 by brim portion 412. In another embodiment, fabric reinforcement layer 430 is configured to at least substantially prevent propagation of tears or perforations in prosthetic cuff 404.

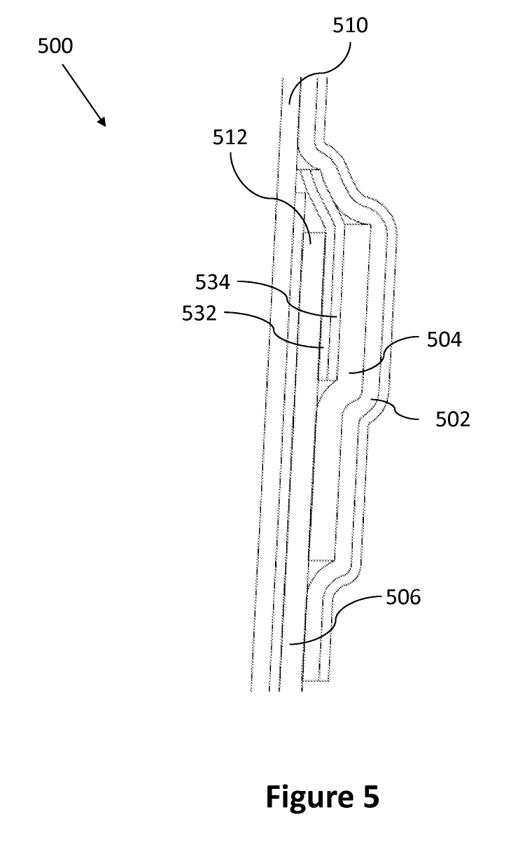

FIG. 5 illustrates a partial view of another example arrangement of a prosthetic sleeve and cuff assembly 500. Assembly 500 comprises a prosthetic sleeve 502, which is oriented radially outward of a prosthetic cuff 504, which is oriented radially outward of a prosthetic socket 506. Assembly 500 further includes a liner 510 oriented radially inward of prosthetic cuff 504 and prosthetic socket 506. Prosthetic socket 506 comprises a brim portion 512.

Prosthetic cuff 504 may comprise an elastomer material. Prosthetic cuff 504 comprises an inner surface and an outer surface, at least a portion of each of which may comprise a tacky elastomer. In one embodiment, prosthetic cuff 504 further comprises a modular cuff portion comprising a modular cuff fabric 532 and a modular cuff elastomer 534. The modular cuff portion may be either separate from prosthetic cuff 504, or integrally connected to prosthetic cuff 504. Modular cuff elastomer 534 may comprise a tacky elastomer. In one embodiment, modular cuff fabric 532 is configured to contact prosthetic socket 506. In another embodiment, modular cuff fabric 532 is configured to contact brim portion 512. In another embodiment, modular cuff fabric 532 is configured to at least partially prevent or mitigate abrasion of prosthetic cuff 504 by brim portion 512. In another embodiment, modular cuff fabric 532 is configured to at least substantially prevent propagation of tears or perforations in prosthetic cuff 504.

While the present application has been illustrated by the description of embodiments thereof, and while the embodiments have been described in considerable detail, it is not the intention to restrict or in any way limit the scope of the appended claims to such detail. Additional advantages and modifications will readily appear to those skilled in the art, having the benefit of the present application. Therefore, the application, in its broader aspects, is not limited to the specific details, illustrative examples shown, or any apparatus referred to. Departures may be made from such details, examples, and apparatuses without departing from the spirit or scope of the general inventive concept.

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. See Bryan A. Garner, A Dictionary of Modern Legal Usage 624 (2d. Ed. 1995). Also, to the extent that the terms “in” or “into” are used in the specification or the claims, it is intended to additionally mean “on” or “onto.” To the extent that the term “selectively” is used in the specification or the claims, it is intended to refer to a condition of a component wherein a user of the apparatus may activate or deactivate the feature or function of the component as is necessary or desired in use of the apparatus. To the extent that the term “operatively connected” is used in the specification or the claims, it is intended to mean that the identified components are connected in a way to perform a designated function. To the extent that the term “horizontal” or “vertical” is used in the specification or the claims, it is intended to mean that the identified components are substantially horizontal or substantially vertical, respectively, when installed in a human that is standing in an upright position. As used in the specification and the claims, the singular forms “a,” “an,” and “the” include the plural. Finally, where the term “about” is used in conjunction with a number, it is intended to include ±10% of the number. In other words, “about 10” may mean from 9 to 11.

1. A prosthetic cuff for use with a prosthetic socket, the prosthetic cuff comprising:
a tubular body portion, comprising an inner surface and an outer surface;
wherein at least a portion of the inner surface includes a tacky elastomer;
wherein at least a portion of the inner surface is configured to contact a prosthetic socket; and
wherein at least a portion of the outer surface includes a tacky elastomer.

2. The prosthetic cuff of claim 1, wherein the tubular body portion comprises at least one of: a thermoplastic elastomer, a urethane, a silicone, a thermoplastic urethane, and a thermoplastic silicone.

3. The prosthetic cuff of claim 1, wherein at least a portion of the inner surface further comprises a fabric reinforcement layer configured to contact a prosthetic socket.

4. The prosthetic cuff of claim 3, wherein at least a portion of the fabric reinforcement layer is further configured to contact a prosthetic socket having a brim portion, at the brim portion.

5. The prosthetic cuff of claim 1, wherein at least a portion of the inner surface is further configured to contact a prosthetic socket having a brim portion, at the brim portion.

6. The prosthetic cuff of claim 1, wherein the tubular body portion further comprises an interior section between the inner surface and the outer surface and wherein at least a portion of the interior section comprises a fabric reinforcement layer.

7. The prosthetic cuff of claim 1, wherein at least a portion of the tubular body portion is impermeable to air.

8. A prosthetic cuff for use with a prosthetic socket, the prosthetic cuff comprising:
a tubular body portion, comprising an inner surface, an outer surface, and an interior section between the inner surface and the outer surface;
wherein at least a portion of at least one of the inner surface and the interior section comprises a fabric reinforcement layer;
wherein at least a portion of the inner surface is configured to contact a prosthetic socket; and
wherein at least a portion of the outer surface includes a tacky elastomer.

9. The prosthetic cuff of claim 8, wherein the tubular body portion comprises at least one of: a thermoplastic elastomer, a urethane, a silicone, a thermoplastic urethane, and a thermoplastic silicone.

10. The prosthetic cuff of claim 8, wherein at least a portion of the inner surface further comprises a tacky elastomer.

11. The prosthetic cuff of claim 8, wherein at least a portion of the inner surface is further configured to contact a prosthetic socket having a brim portion, at the brim portion.

12. The prosthetic cuff of claim 11, wherein at least the portion of one of the inner surface and the interior section comprising the fabric reinforcement layer is configured to contact a prosthetic socket having a brim portion, at the brim portion.

13. The prosthetic cuff of claim 8, wherein at least a portion of the tubular body portion is impermeable to air.

14. The prosthetic cuff of claim 8, wherein the fabric reinforcement layer is configured to stop the propagation of a tear in the tubular body.

15. A prosthetic cuff and sleeve assembly for use with a prosthetic socket, the prosthetic cuff and sleeve assembly comprising:
a cuff comprising a cuff tubular body portion, wherein the cuff tubular body portion comprises a cuff inner surface and a cuff outer surface;
wherein at least a portion of the cuff inner surface is configured to contact a prosthetic socket;
wherein at least a portion of the cuff outer surface includes a tacky elastomer; and
a sleeve comprising a sleeve tubular body portion, wherein the sleeve tubular body portion comprises a sleeve inner surface and a sleeve outer surface;
wherein at least a portion of the sleeve inner surface includes a tacky elastomer.

16. The system of claim 15, wherein at least a portion of the cuff inner surface comprises a tacky elastomer.

17. The system of claim 15, wherein the cuff further comprises a cuff interior section between the cuff inner surface and cuff outer surface, and wherein at least a portion of at least one of the cuff inner surface and the cuff interior section comprises a fabric reinforcement layer.

18. The system of claim 15, wherein the sleeve is positioned radially outwardly of the cuff, and wherein the cuff outer surface and the sleeve inner surface interface is impermeable to air.

19. The system of claim 15, wherein the sleeve outer surface includes a fabric layer.

20. The system of claim 15, wherein the cuff and the sleeve comprise at least one of:
a thermoplastic elastomer, a urethane, a silicone, a thermoplastic urethane, and a thermoplastic silicone.
